(19) United States
(12) Patent Application Publication
(10) Pub. No.: US 2015/0202418 A1
(43) Pub. Date: Jul. 23, 2015

(54) MICRONEEDLE-BASED DEVICES AND METHODS FOR THE REMOVAL OF FLUID FROM A BODY

(71) Applicant: RENEPHRA LIMITED, Manchester (GB)

(72) Inventors: Todd Simon, Manchester (GB); Ian Middleton, Manchester (GB)

(21) Appl. No.: 14/416,460

(22) PCT Filed: Jul. 23, 2013
(86) PCT No.: PCT/GB2013/051959

§ 371 Date: Jan. 22, 2015

(30) Foreign Application Priority Data
Jul. 23, 2012 (GB) 1213073.8

(51) Int. Cl. A61M 37/00; A61F 13/53; A61F 13/15; A61M 5/32

(52) U.S. Cl. CPC A61M 37/0015; A61M 5/3295; A61F 13/53; A61F 13/15203; A61M 2037/0007; A61M 2037/0061; A61F 2013/530481; A61M 2037/0046; A61F 2013/15471; A61F 2013/1552

(57) ABSTRACT

Microneedle-based devices and associated methods for removing fluid from a body are described. The devices incorporate microneedles capable of fluidly linking the body to a high capacity absorbent material capable of absorbing at least 5 times its own weight of said fluid. The microneedles may be conventional hollow microneedles in which the bores are filled with a microporous material, hydrophobic hollow microneedles provided with bores having a hydrophilic lining, hollow hydrogel microneedles or solid microporous microneedles.

### CROSS-REFERENCE TO RELATED APPLICATIONS

This application is a 371 U.S. national stage of International Application No. PCT/GB2013/051959 filed Jul. 23, 2013, which claims the benefit of United Kingdom Application No. GB 1213073.8 filed Jul. 23, 2012, and incorporated herein by reference in their entirety.

### BACKGROUND OF THE INVENTION

The present invention relates to devices for extracting or filtering a fluid, said fluid optionally containing one or more target molecules, such as, but not limited to, common components of interstitial fluid, blood and/or gastrointestinal fluid. The present invention further relates to microneedle devices and absorbent materials which may form part of such a device and methods employing such a device and such materials. The device, associated materials and methods may find application in the medical field, particularly, but not exclusively in the treatment of symptoms such as fluid overload and uraemia arising from conditions such as kidney/renal failure and heart failure.

Native kidneys generate a flow of fluid from the systemic vasculature to the urinary system ending in the bladder prior to voiding. The common and highly generalised view of this function of fluid loss is to rid the body of toxic metabolic waste because in the absence of any renal function death from fluid overload or uraemia ensues within days, uraemia being defined as a medical condition in which kidney function regresses and the kidney fails to excrete into urine the substances that it would otherwise normally have removed, including fluid. As a result of suffering this loss of kidney function excess fluid and uraemic retention products, i.e. substances which are insufficiently removed as a result of the failing kidneys, accumulate. Uraemic toxins are classified as those uraemic retention products which have been shown to exert, typically deleterious, biological or biochemical activity which would not occur if the kidneys were functioning normally.

Another process, which is equally important, is control of body fluid volume and ion balance (Na⁺, Ca²⁺, Cl⁻, PO₄⁻ etc). About 42% of the total body water is extracellular with large variation in the organ distribution of this water—varying from about 13% of total tissue water for skeletal muscle, up to 70% for skin and connective tissue. During conventional dialysis (peritoneal or haemodialysis), excess fluid is removed from the systemic vascular circulation of uraemic patients. The excess fluid is, however, mainly located in the skin and subcutaneous interstitial tissues.

The interstitium is a metabolically active compartment (lactate concentration is higher than plasma), it surrounds cells, maintaining homeostasis and in uraemic individuals, provides a reservoir for extracellular toxins. Unlike the circulatory system, the interstitial albumin concentration is significantly lower than in serum demonstrated both in adipose tissue (15% of serum) and skeletal muscle (27% of serum). A dynamic equilibrium exists between the extracellular interstitial pool and the vascular compartment as demonstrated by conventional dialysis.

Loss of kidney function resulting in end-stage renal failure is a major clinical problem with a wide variety of causes. In the UK, over 37,000 people are receiving renal replacement therapy (RRT) at a cost of £1.5 billion per annum (2% of the total NHS budget). With over 5,000 new additions per year, the UK Renal Registry predicts that the number of patients will rise to 60,000 by 2020. Similar increases in incident patients are expected in the developed healthcare systems in USA and Europe. In the developing world, RRT is highly restricted or absent due to cost and lack of trained healthcare personnel such that renal failure is essentially a death sentence for most (as it was pre-1970 in UK). With the developing economies of China and India able to support improved healthcare for their populations, there is potential to treat renal failure in an additional 2-3 billion population providing the therapy can be delivered in a less technological environment and at cheaper cost than currently available.

The current options for renal replacement therapy (RRT) are predominantly only available in healthcare systems of the developed world.

A first option is kidney transplantation. Although transplantation provides a better treatment and quality of life, with a one year survival rate of 97% compared 84% on dialysis, in the UK only 1,500 kidneys are available annually, with a transplant waiting list of over 5,000 and growing. Those likely to receive a transplant are younger (median age 49 years, with fewer cardiovascular and other comorbidities) than those on dialysis (peritoneal 58, haemodialysis 64 years), which leaves an expanding population of older patients for whom transplantation is not a realistic option.

Current dialysis provision is either haemodialysis or peritoneal dialysis. Haemodialysis involves connecting the patient's blood circulation via a surgically constructed arterio-venous fistula or graft to an external machine that allows removal of low molecular weight metabolites and water across a semi-permeable membrane with return of the “cleansed” blood to the patient. This is predominantly provided in hospital requiring the patient to attend a minimum of 3 days per week (at least 3×4 hour sessions). Significant clinical problems with this modality include failure of vascular access and sepsis and the patient must meet a level of cardiovascular fitness. Quality of life is poor as the patient has to spend 3 days a week in hospital. There is growing evidence of improved patient outcome with frequent or continuous dialysis but this has logistical constraints and is not feasible with current dialysis technology.

Peritoneal dialysis uses the patient's own peritoneal membrane (lining the peritoneal cavity and the visceral organs) as a semi-permeable membrane. With a permanent peritoneal catheter in place, 2 litres of an osmotic solution are in-fused into the peritoneum and after a 4 hour dwell period, the solution is drained out. Low molecular weight metabolites and water from the myriad blood capillaries in the membrane are driven by the osmotic gradient into the in dwelling dialysis solution. This sequence is repeated 3 or 4 times in 24 hour period. Automated versions of this modality allow the patient to connect overnight to a machine that provides frequent flushing of the peritoneal cavity.

Significant clinical problems with this modality include failure of the ultrafiltration function of the membrane and excessive membrane scarring which lead to technique failure.

Congestive Heart Failure (CHF) is an inability of the heart to provide sufficient pump action to maintain blood flow sufficient to meet the needs of the body. Fluid overload is one of the key problems in patients with CHF, whereby excess water and salt accumulate in a patient's body (tissue interstitium) and cause shortness of breath, decreased function of vital organs and swelling of extremities. All this leads to a high rate of hospital admissions of patients with CHF and an increased risk of death.

CHF is a highly prevalent, costly condition that imposes a significant burden on those it affects. Globally, over 26 million people are suffering from Congestive Heart Failure and 2 million new cases are diagnosed every year. This number is expected to grow at 8% annually, mainly due to the aging of the population. The total economic burden of CHF was estimated to be $39.2 billion in 2010 in US alone.

In addition to improving heart's performance, CHF treatment aims to remove excess water and sodium (salt) from the body to achieve fluid balance (euvolemia), relieve symptoms and improve the overall quality of life of patients.

Low salt diet, fluid restriction and diuretics are used to reduce fluid volume. However around 30% of CHF patients experiencing fluid overload do not respond to diuretics. Despite this, many are prescribed large diuretic doses and can suffer from serious adverse effects such as deafness. As a result, many advanced CHF patients are left in a state of chronic fluid retention which leads to increased mortality and morbidity resulting in increased hospital admissions, poor patient performance status and an increased need for drug treatment

Aquapheresis/Ultrafiltration is a relatively new treatment method introduced in 2005 and designed to remove fluid in CHF patients who are resistant to diuretics. It is essentially a simplified haemodialysis and still relies on access to blood. Up until 2008 there were 15,000 patients treated with this method in 250 clinics worldwide however various factors, including the high cost, represent barriers to adoption.

### BRIEF DESCRIPTION OF THE INVENTION

An object of the present invention is to obviate or mitigate one or more of the aforementioned problems.

A first aspect of the present invention provides a fluid extraction or filtration device for removing fluid from a body comprising an array of microneedles in fluid communication with a high capacity absorbent material capable of imbibing at least 5 times the weight of fluid per unit weight of absorbent, wherein at least one of said microneedles is a hollow microneedle, the hollow microneedle defining a tip, a base end and an internal bore connecting an opening in the microneedle to the base end of the microneedle, wherein a microporous material is provided in said internal bore, the microporous material defining an internal flow path for fluid to pass through the microporous material.

In an alternative to the first aspect of the invention, the microneedle device can be used without the high capacity absorbent material. In such an embodiment, the microneedle device comprises at least one hollow microneedle defining a tip, a base end and an internal bore connecting an opening in the microneedle to the base end of the microneedle, wherein a microporous material is provided in said internal bore, the micronporous material defining an internal flow path for fluid to pass through the microporous material.

A second aspect of the present invention relates to a fluid extraction or filtration device for removing fluid from a body comprising an array of microneedles in fluid communication with a high capacity absorbent material capable of imbibing at least 5 times the weight of fluid per unit weight of absorbent, wherein at least one of said microneedles is a hollow microneedle, the hollow microneedle defining a tip, a base end and an internal bore connecting an opening in the microneedle to the base end of the microneedle, wherein the microneedle is fabricated from a hydrophobic material and a surface of the microneedle defining the internal bore is hydrophilic.

In an alternative to the second aspect of the invention, the microneedle device can be used without the high capacity absorbent material. In such an embodiment, the microneedle device comprises at least one hollow microneedle defining a tip, a base end and an internal bore connecting an opening in the microneedle to the base end of the microneedle, wherein the microneedle is fabricated from a hydrophobic material and a surface of the microneedle defining the internal bore is hydrophilic.

A third aspect of the present invention relates to a fluid extraction or filtration device for removing fluid from a body, the device comprising an array of microneedles in fluid communication with a high capacity absorbent material capable of imbibing at least 5 times the weight of fluid per unit weight of absorbent, wherein at least one of said microneedles is a hollow microneedle comprising a swellable material, the hollow microneedle defining a tip, a base end and an internal bore connecting an opening in the microneedle to the base end of the microneedle, the internal bore being fluidly connected to the high capacity absorbent material via the base end of the hollow microneedle.

A fourth aspect of the present invention provides a fluid extraction or filtration device for removing fluid from a body, the device comprising an array of microneedles in fluid communication with a high capacity absorbent material capable of imbibing at least 5 times the weight of fluid per unit weight of absorbent, wherein at least one of said microneedles comprises a microporous material which defines an internal flow path for fluid from the body to pass through the material of the microneedle to the high capacity absorbent material at a flow rate of at least around 0.007 ml/min.

A fifth aspect of the present invention provides an absorbent pouch comprising a high capacity absorbent material capable of imbibing at least 5 times the weight of fluid per unit weight of absorbent retained in an outer skin which comprises a fluid impermeable region and a fluid permeable region to facilitate fluid movement through the outer skin to the absorbent material.

The pores defined by porous materials are often defined according to their size; micropores being less than 2 nm in diameter, mesopores being larger and having diameters up to 50 nm while macropores have diameters larger than 50 nm. Herein references to “microporous” materials should be understood as referring to materials incorporating micropores, mesopores and/or macropores as defined above, the generic term “microporous” being used for the sake of clarity and conciseness. Moreover, it will be appreciated by the skilled person that “microporous materials” will typically contain pores of a range of different sizes, which may all lie within the microporous, mesorporous or macroporous size range, or which may lie in two of these size ranges, or which may lie all three of these size ranges.

The concept of using microneedles for the purpose of delivery of a drug/vaccine into the skin was proposed many years ago but was not realised until relatively recently when microfabrication techniques had developed sufficiently to enable the necessary microstructures to be manufactured. Microneedle arrays penetrating the stratum corneum and entering the epidermis for drug delivery are typically bloodless and painless due to their small dimensions reducing the chance of hitting/stimulating a nerve ending or capillary. The holes created by such microneedles are probably smaller than skin abrasions experienced in daily life. Aspects of the present invention employ the creation of micron sized holes through the dermis to access the interstitial fluid bathing the rich capillary network in the dermis to facilitate transdermal interstitial fluid removal since skin permeability can be increased by orders of magnitude by use of devices according to the various aspects of the present invention set out above.

Whilst the drug delivery industry has focused on needle arrays to deliver drugs from a patch reservoir into the skin (i.e. outside to inside the body), aspects of the present invention relate to the use of microneedles in fluid connection with a high capacity absorbent material to remove components of interstitial fluid such as water, uraemic toxins and/or metabolites from the interstitial skin compartment, that is, to cause a reverse flow of fluid from inside the body to the gel component which will be physically isolated from the interstitial fluid.

The accessibility of the interstitium, the predominant extracellular solute and excess fluid reservoir in fluid overloaded and uraemic individuals, through transdermal microneedles is fundamental to control of interstitial fluid volume and composition. Devices according to the present invention thus provide an important means by which transdermal filtration, purification and/or dialysis of the interstitial fluid can be achieved.

Reference herein to the ‘extraction’ of an amount of fluid can be considered in a similar way to simply ‘removal’ of that amount of fluid, regardless of the amount of fluid being, or intended to be, removed. However, it should be appreciated that reference herein to ‘filtration’ should he interpreted in accordance with the usual way in which this term is used in the (bio)chemical and/or clinical setting. That is, ‘filtration’ refers to the removal of typically relatively large quantities of a fluid (e.g. a biological fluid) from a body. For the avoidance of doubt it will be appreciated by the skilled person that in certain circumstances excess water alone can be regarded as a ‘toxin’ requiring removal from the body. Moreover, by appropriately arranging the device according to the present invention it can be used to selectively remove targeted toxic substances, such as uremic retention products like urea and creatinine, or exogenous toxins, for example during the treatment of poisoning. By selectively removing one or more fluid constituents it will be appreciated that the composition of the fluid remaining in the body after filtration will differ from its original composition. In contrast, a ‘sampling device’ is usually used to obtain a significantly smaller amount of an unmodified fluid (e.g. biological fluid) which is just sufficient to allow appropriate analysis to detect the levels of various constituents, both normal and abnormal, leaving the composition of the remaining fluid unchanged. The selective removal or filtration of excess fluid and uremic retention products resulting from kidney failure for which the devices according to the present invention are eminently suitable, is therefore fundamentally different from merely sampling a small quantity of body fluid to measure the levels of various constituents, such as glucose and/or cholesterol.

A further aspect of the present invention provides a combined fluid extraction and sampling device comprising a fluid extraction or filtration device according to the first, second, third or fourth aspects of the present invention, and sampling means operatively connected to high capacity absorbent material, said sampling means arranged to determine the level of a target species in said fluid.

A related aspect of the present invention provides a method for determining the level of a target species in a sample of fluid extracted from a body, the method comprising extracting said sample from said body using a device according to the above further aspect of the present invention, and analysing said sample of fluid to determine the level of said target species in said sample.

Further aspects of the present invention provide methods for transdermal filtration or purification employing a device according to the first second third or fourth aspects of the present invention, the method comprising contacting said body fluid with said microneedles so that fluid containing a target species flows from said body to said high capacity absorbent material via said microneedles such that said target species are retained in said high capacity absorbent material.

A further aspect of the present invention provides a method for transdermal filtration or purification employing a pouch according to the fifth aspect of the present invention, the method comprising contacting said body fluid with an array of microneedles to establish a fluid path for fluid containing said target species to flow out of said body and placing the pouch in said fluid path so that fluid containing a target species flows from said body to said high capacity absorbent material via said fluid path such that said target species are retained in said high capacity absorbent material.

Another aspect of the present invention provides a method for transdermal filtration employing a device according to the first, second, third or fourth aspects of the present invention, the method comprising contacting said body fluid with said microneedles such that fluid flows from said body to said high capacity absorbent material via said microneedles.

A still further aspect of the present invention provides a method for transdermal filtration employing a pouch according to the fifth aspect of the present invention, the method comprising contacting said body fluid with an array of microneedles to establish a fluid path for fluid to flow out of said body and placing the pouch in said fluid path so that fluid flows from said body to said high capacity absorbent material via said fluid path.

There is further provided a method for renal replacement therapy comprising transdermal filtration employing a device according to the first, second, third or fourth aspects of the present invention, the method comprising contacting said body fluid with said microneedles such that fluid flows from said body to said high capacity absorbent material via said microneedles.

Another aspect of the present invention provides a method for renal replacement therapy comprising transdermal filtration employing a pouch according to the fifth aspect of the present invention, the method comprising contacting said body fluid with an array of microneedles to establish a fluid path for fluid to flow out of said body and placing the pouch in said fluid path such that fluid flows from said body to said high capacity absorbent material via said fluid path.

A yet further aspect of the present invention provides a method for the treatment of uraemia comprising transdermal filtration employing a device according to the first, second, third or fourth aspects of the present invention, the method comprising contacting said body fluid with said microneedles such that fluid containing one or more uraemic toxin flows from said body to said high capacity absorbent material via said microneedles.

Another aspect of the present invention provides a method for the treatment of uraemia comprising transdermal filtration employing a pouch according to the fifth aspect of the present invention, the method comprising contacting said body fluid with an array microneedles to establish a fluid path for fluid to flow out of said body and placing the pouch in said fluid path such that fluid containing one or more uraemic toxins flows from said body to said high capacity absorbent material via said fluid path.

A still further aspect of the present invention provides a method for the treatment of salt and water overload in conditions such as heart failure employing a device according to the first, second, third or fourth aspects of the present invention, the method comprising contacting said body fluid with said microneedles such that fluid containing one or more uraemic toxin flows from said body to said high capacity absorbent material via said microneedles.

Another aspect of the present invention provides a method for the treatment of salt and water overload in conditions such as heart failure employing a pouch according to the fifth aspect of the present invention, the method comprising contacting said body fluid with an array of microneedles to establish a fluid path for fluid to flow out of said body and placing the pouch in said fluid path such that fluid containing one or more uraemic toxin flows from said body to said high capacity absorbent material via said fluid path.

In respect of the above defined aspects of the present invention it is preferred that said target species is selected from the group consisting of water, a uraemic toxin, a metabolic product, a salt and an ion. Alternatively, said target species may be selected from the group consisting of Retinol Binding Protein, Beta-2-Microglobulin, Parathyroid hormone, Adrenomedullin, Atrial Natriuretic Peptide, Asymmetric dimethylarginine, Indole-3-Acetic Acid, Uric Acid, Homocysteine, Creatine, Creatinine, P-Cresol, Oxalate, Urea and Phosphate.

In a preferred embodiment of the present invention there is provided a wearable, continuous slow mode of filtration for example, for the purpose of purification and/or dialysis, that accesses the interstitial fluid through the skin in order to remove target species such as, but not limited to, low molecular weight metabolites and/or water. An exemplary embodiment of the device according to the third aspect of the present invention is shown in FIG. 1, which will be described in greater detail below.

A still further aspect of the present invention provides a method of treating oedema by the removal of interstitial fluid from an area of oedema in a body, the method comprising the insertion of one or more arrays of microneedles into tissue swollen as a result of oedema, removing the microneedles to establish a fluid path for fluid within the oedema to flow from the swollen tissue and then placing a high capacity absorbent material capable of imbibing at least 5 times the weight of fluid per unit weight of absorbent in the fluid flow path such that fluid within the swollen tissue flows from said tissue to said high capacity absorbent material via said fluid flow path at a flow rate of at least around 0.007 ml/mm.

Application of the high capacity absorbent material over the punctures made by the microneedles would have been expected by the skilled person to result in the holes closing rapidly as the skin surface reseals and thus limiting the amount of fluid that could be removed. Surprisingly, it has been determined that if the absorptive rate and capacity of the absorbent material is sufficiently high the holes will remain open and enable fluid to be removed over a prolonged timespan of hours or even a day or more. The high capacity absorbent is configured to remove fluid from the body at a flow rate of at least around 0.007 ml/min and to he capable of imbibing at least 5 times the weight of fluid per unit weight of absorbent. Any article designed to capture fluid in this way may be used as the high capacity absorbent material, including adhesive foam dressings, alginate fibres, carboxymethyl cellulose fibres.

Another aspect of the present invention provides a method of treating oedema by the removal of interstitial fluid from an area of oedema in a body, the method comprising the insertion of one or more arrays of microneedles into tissue swollen as a result of oedema, removing the microneedles to establish a fluid path for fluid within the oedema to flow from the swollen tissue and then placing a pouch according to the fifth aspect of the present invention in the fluid flow path such that fluid within the swollen tissue flows from said tissue to said high capacity absorbent material via said fluid flow path.

A preferred embodiment of this aspect of the invention is the use of an absorbent pouch consisting of superabsorbent hydrogel (for example, lightly crosslinked sodium acrylate) particles contained within an outer skin comprised of a non-woven mesh that contains superabsorbent fibrous material (such as Oasis fibres, available from Technical Absorbents Limited). The non-woven mesh may contain other synthetic fibres that allow it to be sealed by conventional techniques such as thermal bonding, ultrasonic welding and infrared welding. The pouch may be held in place over the puncture site with the aid of adhesive tape or an adhesive coated transparent conformable film. This construct has the advantage over the use of commercially available dressings to have much higher absorbent capacity per unit area thereby minimizing the need to change the pouch and risking closure of the puncture holes.

The use of microneedle arrays, which may comprise solid microneedles, for the purpose of producing punctures of regions of oedema is preferred over the use of a conventional syringe needle as the size of the microneedles minimises pain and trauma during the puncture process by not penetrating the subcutaneous capillary bed. Accordingly the microneedle height should be up to around 1000 μm or less, preferably around 700 μm or less and be capable of creating holes in the stratum corneum of up to around 1000 μm.

It will be appreciated that a vacuum suction device could be used in combination with any of the above-defined aspects of the present invention to accelerate the transdermal extraction or removal of water, uraemic toxins, metabolic products, ions and/or salts, which may find particular application in the treatment of fluid overload or uraemia arising from, for example, heart failure and/or renal failure.

An example of a protocol which has been successfully used by the applicant to achieve the abovementioned methods of transdermal filtration or purification, renal replacement therapy, treating uraemia, salt and water overload and oedema, through use of a vacuum suction device is shown below:
1. An appropriate skin antiseptic is applied to the insertion site on the skin of the patient.
2. A microneedle array is applied to the skin. This can be done using a microneedle applicator.
3. The rear of the microneedle is assessed and fluid flow is noted.
4. If no fluid is observed on the skin, the microneedle array can be reapplied.
5. If fluid is observed, the microneedles are removed from the skin and a pre-weighed high capacity absorbent material is applied to the wound site.
6. A piece of black foam is applied to the high capacity absorbent material.
7. A transparent film dressing is used to seal the high capacity absorbent material and foam to the skin.
8. An incision is made on the film dressing and a valve adapter for a vacuum pump is placed over the incision.
9. Tubing is connected to the valve adapter and the vacuum pump.
10. The vacuum pump is activated to provide a suction pressure of 75-250 mmHg.
11. The vacuum is left in place for up to 2 hours.
12. The high capacity absorbent material is removed and weighed.
13. Steps 5-12 are repeated as required.
14. An appropriate skin antiseptic is applied to the wound site.

Suitable high capacity absorbent materials which could be used in the abovementioned protocol include absorbent wound dressings such as those available from Crawford Healthcare manufactured under the name KerraMax Care™. Suitable vacuum dressing systems which could be used in the abovementioned protocol include V.A.C.® GranuFoam™ dressings available from KCI Medical.

A potential drawback to this aspect of the invention is that allowing interstitial fluid to escape from direct punctures in the skin surface may lead to maceration in the vicinity of the holes. Although skin maceration may be viewed as an acceptable consequence of fluid removal from patients suffering with diminished renal function, it should be avoided if possible. This disadvantage may be overcome by use of hollow microneedles in fluid communication with an absorbent gel matrix, as described in WO 2009098487, wherein the hollow microneedles act as a conduit for fluid and the outer skin surface does not come into contact with any interstitial fluid thereby avoiding maceration. While the device described in WO 2009098487 may function adequately in areas of oedematous tissue having a high positive interstitial pressure, if the hollow microneedles are fabricated from hydrophobic engineering plastics, such as polyether-ether-ketone (PEEK), polycarbonate (PC), or polyimide, transport of fluid will be impaired by poor capillary action. This is due to the poor wettability of the hydrophobic material by interstitial fluid, as may be deduced from the equation for the height (h) of a liquid in a capillary that is given by:

h=2γ cos θ/ρgr

where γ is the liquid air surface tension, θ is the contact angle, ρ is the liquid density, g is the gravitational force constant and r is the radius of the capillary.

For water the contact angle with PEEK is 71° giving cos θ=0.33, while with serum the contact angle with PEEK is 85° giving cos θ=0.08. For a completely wettable surface the contact angle is 0°, giving cos θ=1. Set out below are various aspects of the present invention which describe modified microneedles designed to obtain satisfactory transport of interstitial fluid.

In the device according to the first aspect of the present invention a high capacity absorbent material capable of imbibing at least 5 times the weight of fluid per unit weight of absorbent is provided in fluid communication with at least one hollow microneedle having a tip, a base end and internal bore in which the internal bore of the hollow microneedle is provided with microporous material defining an internal flow path for fluid flow.

In order to facilitate transport of interstitial fluid from the surface of the microporous material to the surface of the absorbent material it is preferable to extend contact between the two surfaces by providing microporous material through to the base end of the at least one microneedle. The microporous material in the bore of the hollow microneedle is preferably a separate material to the high capacity absorbent material.

The base end of the at least one hollow microneedle may be directly connected to the high capacity absorbent material such that the internal bore of the microneedle is directly fluidly connected to the high capacity absorbent material. The device may further comprise an intermediate fluid transport layer adjacent to the base end of the hollow microneedle Which fluidly connects the internal bore of the hollow microneedle to the high capacity absorbent material. The intermediate fluid transport layer may take any convenient form, but may, for example, be in the form of a sheet material that is capable of effecting fluid transport by wicking.

The high capacity absorbent material may conveniently be provided in a pouch or bag incorporating a fluid permeable region through which fluid can pass from outside the pouch to the high capacity absorbent material. The structure of the pouch or bag may be provided by an outer skin which incorporates one or more fluid impermeable regions in addition to the fluid permeable region.

The microporous material is preferably adapted such that fluid from the body can pass along said internal flow path at a rate of at least around 0.007 ml/min, more preferably a flow rate of at least around 0.035 ml/min, and still more preferably at a flow rate of at least around 0.07 ml/min. The microporous material may be such that fluid from the body can pass along said internal flow path at a rate of at least around 1.4 ml/min, more preferably up to around 0.7 ml/min and yet more preferably up to around 0.35 ml/min. It is preferred that the microporous material is adapted such that fluid from the body can pass along said internal flow path at a rate of around 0.007 to around 1.4 ml/min, more preferably around 0.035 to around 0.7 ml/min.

The microporous material may be a polymer, hydrogel, metal oxide, ceramic, or a composite. Manufacturing routes of such porous matrices will be known to those skilled in the art.

The microporous material provided within the internal bore preferably comprises one or more polymeric materials. Suitable microporous materials may comprise open cell hydrophilic polyurethane foam, polyurethane mixed with impact modified polystyrene, polyethylene glycol and polyvinyl alcohol, polyethylene glycol and dextran, or polyethylene glycol and poly(dimethylsiloxane).

In a preferred embodiment the microporous material is derived from a coagulated solution via a phase inversion process. The solution may comprise polyurethane, polyether sulfone or silica.

The microporous material ay be a cryogel, xerogel or aerogel, such as a silica produced by a sol gel route.

The microporous material may have a porosity of around 10 to 99.9%, more preferably around 40 to 70%. Preferred xerogel silica-based materials possess a porosity of around 25% and incorporate pore sizes of around 1 to 10 nm.

Manufacture of porous media may be achieved using, for example, tetraethyl orthosilicate (TEOS). The microporous material may be disposed in the bore, in a process separate to the production of the hollow microneedles. A preferred means of providing a microporous silica to fill the internal bore of a hollow hydrophobic microneedle is by the use of a colloidal silica dispersion that may be coagulated by addition of a solution of an inorganic salt such as sodium or potassium chloride, followed by insertion of the coagulated silica into the bores of the microneedles and drying to produce a filled microneedle array. The concentration of colloidal silica that may be used for this purpose may vary from 30% to 50% of silica, with the latter being a preferred concentration. The concentration of the inorganic salt solution to be used for coagulating the colloidal silica will vary depending upon the nature of the salt and concentration of colloidal silica to be used. Thus a 50% w/w colloidal silica solution may be coagulated by addition of 1.5% w/w sodium chloride solution in a ratio of 2 parts colloidal silica solution to 1 part sodium chloride solution. If potassium chloride is substituted for sodium chloride a lower concentration of potassium chloride or alternatively a higher ratio of colloidal silica to inorganic salt may be required to bring about satisfactory gelation and formation of a microporous silica sol filled needle array.

The opening in the at least one hollow microneedle is preferably defined at or adjacent to the tip of the microneedle, or is defined by a sidewall in between the tip and base end of the hollow microneedle. The opening may have any appropriate diameter and may be of any suitable shape. The opening can be of any appropriate size or suitable shape and may extend to a size up to 500 μm depending on the size and shape of the needle. It is preferred that the opening has a diameter of up to around 250 μm, more preferably around 50 μm to 150 μm, most preferably around 100 μm. While the opening may be a substantially circular hole, in a preferred embodiment the opening is an oval slot. The microneedles are preferably designed to penetrate the stratum corneum. Providing at least one of the microneedles with an opening in a sidewall of the microneedle is advantageous since it avoids the microneedle becoming blocked during and/or following insertion, which could otherwise hinder or even prevent fluid flow from the interstitial skin compartment to the high capacity absorbent material.

The or each hollow microneedle may define two or more of said internal bores. Where more than one internal bore is present, each bore may link a separate, dedicated opening to the base end of the microneedle. Alternatively, two or more internal bores may link a single opening to the base end of the microneedle. As a further alternative, one or more bores may link one opening to the base end of the microneedle, while one or more other bores may link a different opening to the base end of the microneedle.

In embodiments incorporating two or more internal bores, one or more of the internal bores may be microporous material. In a preferred embodiment, the hollow microneedle defines two or more of said internal bores and two or more of said internal bores are provided with a microporous material. The or each internal bore may be partially filled or entirely filled with the microporous material.

In a preferred embodiment of this aspect of the invention the microporous material, such as silica, may extend beyond the internal bores of the microneedles to the base end of the microneedle array, for example, in the form of a series of trenches that provide greater surface contact between the absorbent material in fluid communication with the microporous material.

An alternative means of providing a microporous material to be provided in the internal bore of a hydrophobic hollow microneedle array is by use of a formulation that is a modification of a preferred coating system of porous silica particles in a hydrophilic polymer binder for hollow hydrophobic microneedles as will be described in relation to the second aspect of the invention. In order for a system of this type to function to provide a microporous material to be provided in the internal bore of a hydrophobic hollow microneedle the concentration of the hydrophilic polymer binder and the ratio of silica to binder may be increased over that specified for the coating formulation used in the second aspect. Accordingly if using pHEMA as a binder the amount used may be around 8 to 15% w/w, more preferably around 9 to 12% w/w. The ratio of the binder to the porous polar particles may not be less than 1:5. The porous silica particles in the hydrophilic polymer binder may extend from the internal bore of the at least one microneedle to the base end of the microneedle to form a film providing greater surface contact between the absorbent material in fluid communication with the sol gel silica. The bore of the at least one microneedle may be increased in size to allow a greater surface area to be in contact with the fluid bed.

The height of the or each hollow microneedle may be selected for the particular application, and may need to be sufficiently high to provide an inserted portion and an uninserted portion, that is, one or more of the hollow microneedles may need to be high enough such that a first portion of the or each hollow microneedle can reside within the body and a second portion can reside outside the body. The height of one or more of the hollow microneedles may be around 1 μm to 1 mm or around 50 μm to 900 μm. More preferably around 300 μm to 900 μm, still more preferably around 500 μm to around 800 μm, and yet more preferably around 600 μm to 700 μm. In further preferred embodiments one of more of the hollow microneedles preferably possesses a height of up to around 700 μm, more preferably up to around 650 μm. In further preferred embodiments, one or more of the hollow microneedles possesses a height in the range of around 550 μm to around 700 μm, and most preferably a height of around 650 μm.

The cross-sectional dimension of the or each hollow microneedle may be around 10 nm to 1 mm, around 100 μm to 500 μm, or around 300 μm to 400 μm. Preferably the or each hollow microneedle possesses a maximum outer cross-sectional diameter of around 400 μm.

The or each hollow microneedle may comprise or be formed from any suitable material, such as a polymeric material, for example poly(ethylene-ether-ketone), polycarbonate, silica or a metallic material.

In the device according to the second aspect of the present invention a high capacity absorbent material capable of imbibing at least 5 times the weight of fluid per unit weight of absorbent is provided in fluid communication with at least one hollow microneedle fabricated from a hydrophobic material in which a surface of the microneedle defining the internal bore is hydrophilic.

The hydrophilic surface(s) may be provided by the application of a hydrophilic coating to the or each surface of the internal bore, or may be created by derivatising the or each surface with a suitable chemical group which is itself hydrophilic, or which can be subsequently modified to be rendered hydrophilic.

In order to facilitate transport of interstitial fluid from the surface of the coating to the surface of one or more absorbent materials capable of imbibing fluid at a high absorptive rate it is preferable to extend contact between the both surfaces by extending the hydrophilic region to the base end of the at least one microneedle.

The hydrophilic surface is preferably configured to reduce the contact angle of the surface of the internal bore of the microneedle(s) with interstitial fluid to around 0°. Coatings administered by any convenient technique, such as plasma deposition, dip coating, spraying etc., may be used to deposit an appropriate coating.

In a preferred embodiment the hydrophobic material is a polymeric material, such as polyether-ether-ketone (PEEK) of polycarbonate. The hydrophilic surface of the internal bore may comprise a polymeric material or porous polar particles, such as silica particles. The porous polar particles may be embedded within a hydrophilic polymer binder. Any appropriate binder may be employed taking into account various factors, such as the nature of the hydrophilic material and the nature of the surface to which the hydrophilic material is to be bound. A suitable binder, particularly, but not exclusively for use with silica particles is poly(2-hydroxy ethyl methacrylate) (polyHEMA). Another suitable binder, particularly, but not exclusively for use with silica particles, is the hydrophilic polyurethane Tecogel (Velox Ltd) in alcoholic solution. The amount of binder should be selected to suit the particular application. It may be appropriate to use the binder in an amount of around 1 to 8% w/w, more preferably around 3 to 6% w/w. Another suitable binder is a linear polysaccharide, such as chitosan. A suitable amount of a binder of this kind may be around 0.5 to 3% w/w, more preferably around 1 to 2% w/w. The porous polar particles are preferably silica particles such as the Syloid (Grace Ltd) range of silica particles. The ratio of the binder to the porous polar particles may be around 2:1 to 1:5, more preferably around 1:2 to 1:4.

The base end of the at least one hollow microneedle may be directly connected to the high capacity absorbent material such that the internal bore of the microneedle is directly fluidly connected to the high capacity absorbent material. The device may further comprise an intermediate fluid transport layer adjacent to the base end of the hollow microneedle which fluidly connects the internal bore of the hollow microneedle to the high capacity absorbent material. The intermediate fluid transport layer may take any convenient form, but may, for example, be in the form of a sheet material that is capable of effecting fluid transport by wicking.

The high capacity absorbent material may conveniently be provided in a pouch or bag incorporating a fluid permeable region through which fluid can pass from outside the pouch. to the high capacity absorbent material. The structure of the pouch or bag may be provided by an outer skin which incorporates one or more fluid impermeable regions in addition to the fluid permeable region.

The device is preferably configured to remove fluid from the body at a flow rate of at least around 0.007 ml/mm. More preferably the device is configured to remove fluid from the body at a flow rate of at least around 0.035 ml/min, and still more preferably at a rate of at least around 0.07 ml/min. The device may be configured to remove fluid from the body at a flow rate of up to around 1.4 ml/min, more preferably up to around 0.7 ml/min and yet more preferably up to around 0.35 ml/min. It is preferred that the device is configured to remove fluid from the body at a flow rate in the range of around 0.007 to around 1.4 ml/min, more preferably around 0.035 to around 0.7 ml/min.

The opening in the at least one hollow microneedle is preferably defined at or adjacent to the tip of the microneedle, or is defined by a sidewall in between the tip and base end of the hollow microneedle. The opening may have any appropriate diameter and may be of any suitable shape. The opening can be of any appropriate size or suitable shape and may extend to a size up to 500 μm depending on the size and shape of the needle. It is preferred that the opening has a diameter of up to around 250 μm, more preferably around 50 μm to 150 μm, most preferably around 100 μm. The microneedles are preferably designed to penetrate the stratum corneum. Providing at least one of the microneedles with an opening in a sidewall of the microneedle is advantageous since it avoids the microneedle becoming blocked during and/or following insertion, which could otherwise hinder or even prevent fluid flow from the interstitial skin compartment to the high capacity absorbent material.

The or each hollow microneedle may define two or more of said internal bores. Where more than one internal bore is present, each bore may link a separate, dedicated opening to the base end of the microneedle. Alternatively, two or more internal bores may link a single opening to the base end of the microneedle. As a further alternative, one or more bores may link one opening to the base end of the microneedle, while one or more other bores may link a different opening to the base end of the microneedle.

In embodiments incorporating two or more internal bores, one or more of the internal bores may be defined by a surface provided with a hydrophilic coating. In a preferred embodiment, the hollow microneedle defines two or more of said internal bores and surfaces of two or more of said internal bores are provided with a hydrophilic coating.

The height of the or each hollow microneedle may be selected for the particular application, and may need to be sufficiently high to provide an inserted portion and an uninserted portion, that is, one or more of the hollow microneedles may need to be high enough such that a first portion of the or each hollow microneedle can reside within the body and a second portion can reside outside the body. The height of one or more of the hollow microneedles may be around 1 μm to 1 mm or around 50 μm to 900 μm. More preferably around 300 μm to 900 μm, still more preferably around 500 μm to around 800 μm, and yet more preferably around 600 μm to 700 μm. In further preferred embodiments one of more of the hollow microneedles preferably possesses a height of up to around 700 μm, more preferably up to around 650 μm. In further preferred embodiments, one or more of the hollow microneedles possesses a height in the range of around 550 μm to around 700 μm, and most preferably a height of around 650 μm.

The cross-sectional dimension of the or each hollow microneedle may be around 10 nm to 1 mm, around 100 μm to 500 μm, or around 300 μm to 400 μm. Preferably the or each hollow microneedle possesses a maximum outer cross-sectional diameter of around 400 μm.

In the device according to the third aspect of the present invention a high capacity absorbent material capable of imbibing at least 5 times the weight of fluid per unit weight of absorbent is provided in fluid communication with at least one hollow microneedle having a tip, a base end and internal bore in which the hollow microneedle comprises a swellable material.

As the material swells the contact angle tends to zero giving enhanced capillary transport of interstitial fluid through the hollow microneedles. The absorptive rate of the high capacity absorbent materials should be greater than that of the material from which the hollow microneedle is fabricated in order to facilitate fluid flow through the hollow microneedle. The device is preferably configured to remove fluid from the body at a flow rate of at least around 0.007 ml/min. More preferably the device is configured to remove fluid from the body at a flow rate of at least around 0.035 ml/min, and still more preferably at a rate of at least around 0.07 ml/min. The device may be configured to remove fluid from the body at a flow rate of up to around 1.4 ml/min, more preferably up to around 0.7 ml/min and yet more preferably up to around 0.35 ml/min. It is preferred that the device is configured to remove fluid from the body at a flow rate in the range of around 0.007 to around 1.4 ml/min, more preferably around 0.035 to around 0.7 ml/min.

The opening in the at least one hollow microneedle is preferably defined at or adjacent to the tip of the microneedle, or is defined by a sidewall in between the tip and base end of the hollow microneedle. The opening may have any appropriate diameter and may be of any suitable shape. The opening can be of any appropriate size or suitable shape and may extend to a size up to 500 μm depending on the size and shape of the needle. It is preferred that the opening has a diameter of up to around 250 μm, more preferably around 50 μm to 150 μm, most preferably around 100 μm. The microneedles are preferably designed to penetrate the stratum corneum. Providing at least one of the microneedles with an opening in a sidewall of the microneedle is advantageous since it avoids the microneedle becoming blocked during and/or following insertion, which could otherwise hinder or even prevent fluid flow from the interstitial skin compartment to the high capacity absorbent material.

A preferred family of swellable materials that may be used is polymer hydrogels. These materials are capable of absorbing fluid. Examples of suitable polymer hydrogels include crosslinked poly ethylene glycol (PEG), acrylate and methacrylate derivatives of polyethylene glycol, polyurethanes having soft segment structures containing polyethylene glycol, poly acrylamide (PAm), polyacrylate salts such as sodium or potassium polyacrylate, polyvinyl pyrrolidone, poly 2-hydroxy ethyl methacrylate (pHEMA), and any derivative or copolymer of said hydrogels. Other hydrogels may be based on crosslinked natural polysaccharides or semi-synthetic cellulose derivatives such as gellan gum, guar gum, xanthan gum, alginate, chitosan, carboxy methyl cellulose, hydroxyl ethyl cellulose, hydroxyl ethyl methyl cellulose, hydroxyl propyl cellulose, hydroxyl propyl methyl cellulose These materials may be crosslinked alone or in combination with others listed above by reaction with appropriate multifunctional crosslinking agents at elevated temperature. A wide range of crosslinking agents may be used for this purpose including divinyl substituted compounds, such as ethylene dimethacrylate or divinyl benzene, melamine formaldehyde resins, polymers and copolymers of N-methylol acrylamide, and citric acid. Methods of manufacturing swellable microneedles may involve thermoforming, moulding, machining, lithography, or etching.

The base end of the at least one microneedle may be directly connected to the high capacity absorbent material such that the internal bore of the microneedle is directly fluidly connected to the high capacity absorbent material. The device may further comprise an intermediate fluid transport layer adjacent to the base end of the hollow microneedle which fluidly connects the internal bore of the hollow microneedle to the high capacity absorbent material. The intermediate fluid transport layer may take any convenient form, but may, for example, be in the form of a sheet material that is capable of effecting fluid transport by wicking.

The or each hollow microneedle may define two or more of said internal bores. Where more than one internal bore is present, each bore may link a separate, dedicated opening to the base end of the microneedle. Alternatively, two or more internal bores may link a single opening to the base end of the microneedle. As a further alternative, one or more bores may link one opening to the base end of the microneedle, while one or more other bores may link a different opening to the base end of the microneedle.

The height of the or each hollow microneedle may be selected for the particular application, and may need to be sufficiently high to provide an inserted portion and an uninserted portion, that is, one or more of the hollow microneedles may need to be high enough such that a first portion of the or each hollow microneedle can reside within the body and a second portion can reside outside the body. The height of one or more of the hollow microneedles may be around 1 μm to 1 mm or around 50 μm to 900 μm. More preferably around 300 μm to 900 μm, still more preferably around 500 μm to around 800 μm, and yet more preferably around 600 μm to 700 μm. In further preferred embodiments one or more of the hollow microneedles preferably possesses a height of up to around 700 μm, more preferably up to around 650 μm. In further preferred embodiments, one or more of the hollow microneedles possesses a height in the range of around 550 μm to around 700 μm, and most preferably a height of around 650 μm.

The cross-sectional dimension of the or each hollow microneedle may be around 10 nm to 1 mm, around 100 μm to 500 μm, or around 300 μm to 400 μm. Preferably the or each hollow microneedle possesses a maximum outer cross-sectional diameter of around 400 μm.

The fourth aspect of the present invention provides a fluid extraction or filtration device comprising an array of microneedles in fluid communication with a high capacity absorbent material capable of imbibing at least 5 times the weight of fluid per unit weight of absorbent. At least one of said microneedles comprises a microporous material configured such that fluid can pass through the material to the high capacity absorbent material at a flow rate of at least around 0.007 ml/min.

The device is preferably configured to remove fluid from the body at a flow rate of at least around 0.035 ml/min, and still more preferably at a rate of at least around 0.07 ml/min. The device may be configured to remove fluid from the body at a flow rate of up to around 1.4 ml/min, more preferably up to around 0.7 ml/min and yet more preferably up to around 0.35 ml/min. It is preferred that the device is configured to remove fluid from the body at a flow rate in the range of around 0.007 to around 1.4 ml/min, more preferably around 0.035 to around 0.7 ml/min.

The height of the or each microporous microneedle may be selected for the particular application, and may need to be sufficiently high to provide an inserted portion and an uninserted portion, that is, one or more of the microporous microneedles may need to be high enough such that a first portion of the or each microneedle can reside within the body and a second portion can reside outside the body. The height of one or more of the microporous microneedles may be around 1 μm to 1 mm or around 50 μm to 900 μm. More preferably around 300 μm to 900 μm, still more preferably around 500 μm to around 800 μm, and yet more preferably around 600 μm to 700 μm. In further preferred embodiments one or more of the microporous microneedles preferably possesses a height of up to around 700 μm, more preferably up to around 650 μm. In further preferred embodiments, one or more of the microporous microneedles possesses a height in the range of around 550 μm to around 700 μm, and most preferably a height of around 650 μm.

The cross-sectional dimension of the or each microporous microneedle may be around 10 nm to 1 mm, around 100 μm to 500 μm, or around 300 μm to 400 μm. Preferably the or each microporous microneedle possesses a maximum outer cross-sectional diameter of around 400 μm.

Materials that may be used to fabricate microneedle arrays may be blends of 2 or more polymers that undergo phase separation to generate microporous channels throughout the microneedle structure. Many combinations of polymers may be used to develop phase separated channels within a composite structure, examples of such combinations of polymers are blends of polyurethane with impact modified polystyrene, polystyrene and poly methyl methacrylate, polyethylene oxide and polyvinyl alcohol and polyethylene glycol and polydimethyl siloxane. An alternative approach to producing a microporous phase separated structure is to fabricate microneedles from highly crosslinked polymers such as polystyrene crosslinked with divinyl benzene or to use a porometric solvent in the fabrication process in the same manner in which hollow fibre membranes are produced from polyether sulphone by phase inversion from dimethyl suphoxide and water.

The microporous material may be a cryogel, xerogel or aerogel, such as a silica sol gel. The microporous material may have a porosity of around 10 to 99.9%, more preferably around 20 to 50%. Preferred xerogel silica-based materials possess a porosity of around 25% and incorporate pore sizes of around 1 to 100 nm.

To reduce the possibility of maceration of the skin surface in the vicinity of the site of insertion of the microneedle arrays that may be maintained in place for prolonged periods, devices according to the third and fourth aspects of the present invention may be used which provide hollow microneedles modified to enhance the capillary flow of fluid through the bores of the microneedles.

The fifth aspect of the present invention provides an absorbent pouch comprising a high capacity absorbent material capable of imbibing at least 5 times the weight of fluid per unit weight of absorbent. The absorbent material is retained in an outer skin which comprises a fluid impermeable region and a fluid permeable region to facilitate fluid movement through the outer skin to the absorbent material.

It will be appreciated that where reference is made herein to a “high capacity absorbent material”, this does not exclude the presence of two or more different types of absorbent material, or two or more discrete sections of pockets of absorbent material, provided at least one of them is a high capacity absorbent material capable of imbibing at least 5 times the weight of fluid per unit weight of the high capacity absorbent material.

In a preferred embodiment the outer skin may comprise a first lateral wicking membrane layer that can provide fluid communication between the microneedles of the devices according to the first to fourth aspects of the present invention and the absorbent material held within the pouch. The fluid impermeable region(s) of the outer skin of the pouch may comprise a conformable film, which may be transparent, to prevent leakage of fluid swollen absorbent material from the pouch. The first lateral wicking layer may be sealed to the outer conformable film layer by various techniques, such as thermal bonding, ultrasonic welding, vibration welding, adhesive bonding or infrared welding. In a preferred embodiment the outer conformable film layer may extend over a surface of the first lateral wicking layer such that an area of said first wicking layer remains exposed in order to ensure intimate contact and good fluid transmission between the rear of the microneedle array and the wicking layer. The first wicking layer may be connected to the rear of the perimeter of the base end of the microneedle array by any suitable means, such as a pressure sensitive adhesive. The first lateral wicking layer may be constructed from a variety of materials including high absorbency cellulosic membranes, such as tissue paper or filter paper, alternatively woven meshes coated with a wicking medium such as polyester woven mesh coated with a hydrophilic polymer binder containing silica particles may be used or a non-woven fibrous felt containing superabsorbent fibres. The conformable film layer is preferably constructed from an elastomeric polymer such as natural rubber, nitrile rubber, chloroprene rubber, polyurethane, styrene butadiene styrene block copolymers or others that will be known to those skilled in the art. In order to ensure good fixation of the pouch to a patient's skin while in use, areas of the outer conformable film layer that lie proximal to the skin of the patient may be coated with a pressure sensitive adhesive and optionally covered by a siliconised release liner that may be removed after insertion of the microneedles thus allowing the ends of the microneedles to be firmly anchored to the skin surface and to act as a barrier to bacterial invasion of the punctured skin.

The high capacity absorbent material may comprise one or more absorbent materials capable of imbibing fluid at a high absorptive rate such that the overall absorptive capacity of the materials may be 5-35 times the weight of fluid per unit weight of absorbent, more preferably 5-15 times or 15-25 times the weight of fluid per unit weight of absorbent, and most preferably 25-35 times the weight of fluid per unit weight of absorbent. The absorbent material may be secured in place using adhesive tape or an adhesive coated transparent conformable film. Should the maximum absorptive capacity be approached the absorbent material may be removed and replaced by another. Not only is the absorptive capacity of the absorbent material important, so to is the rate at which fluid is transferred from the holes in oedematous skin tissue or microneedle arrays to the absorbent material since this determines the efficacy of interstitial fluid removal. Thus fluid from the body should pass through holes in oedematous skin tissue or the microneedles to the high capacity absorbent material at a flow rate in the range of around 0.007 to around 1.4 ml/min, more preferably around 0.035 to around 0.7 ml/min.

The absorbent material held within the pouch may comprise superabsorbent polymer particles, hydrogel particles, porous silica particles, materials comprising inorganic complexes such as clay particles, in particular smectite clay particles, activated carbon particles or mixtures thereof. A mixture of 2 parts of fine powder superabsorbent particles with one part of porous silica particles may be used to capture interstitial fluid and hold it within the pouch. The absorbent material, preferably in the form of particles which may be of varying composition, may be provided in one or more compartments defined within the pouch separated by second and, optionally further, lateral wicking layers to promote rapid fluid transfer between the absorbent material in each compartment. In a preferred embodiment, a first compartment of the pouch may comprise superabsorbent and silica particles, while a second compartment may contain clay and activated charcoal particles. In certain cases in may be preferable to have activated charcoal present within the pouch as a fibrous cloth layer. A further preferred embodiment comprises the aforementioned two compartments in combination with a third compartment containing a mixture of superabsorbent particles, silica particles, clay particles and/or activated charcoal particles.

In a preferred embodiment of the device according to the first, second, third and/or fourth aspects of the present invention, the device is configured such that, in use, when said at least one microneedle contacts the body fluid, the high capacity absorbent material is physically isolated from the fluid. The high capacity absorbent material can be located inside or outside the body. It is preferred that the high capacity absorbent material is not in direct physical contact with the interstitial fluid, although it will be appreciated that the high capacity absorbent material is, of course, in fluid communication with the interstitial fluid. While the body to which the device is applied may be any type of physical body retaining a fluid, the body is preferably a human or animal body. It is further preferred that the device is configured such that, in use, when said at least one microneedle contacts the fluid, the high capacity absorbent material is located outside the body.

Preferably a first region of said at least one microneedle is arranged for contacting the fluid and the base end of said at least one microneedle is connected to the high capacity absorbent material. In this way, the region of the microneedles that contacts the fluid is separated or spaced apart from the base end that contacts the high capacity absorbent material, which enables the high capacity absorbent material to be physically isolated from the fluid.

The high capacity absorbent material employed in the first, second, third and/or fourth aspects of the present invention is preferably configured to retain said fluid after its removal from the body. If desired, the retained fluid can then be sampled to measure the amount of one or more of its constituents and/or treated in any suitable way to remove any of its constituents. It will be appreciated that where the absorbed fluid is held permanently within the high capacity absorbent material then it will be necessary to periodically exchange saturated material for new, dry material. Otherwise, the high capacity absorbent material can be subjected to some form of treatment to remove the retained fluid and make the device ready for further use.

The body is preferably a human or animal body and the fluid extraction or filtration device according to the first, second, third and/or fourth aspects of the present invention is preferably arranged to effect the selective removal of one or more toxins from said human or animal body.

It is preferred that the device is arranged to effect the selective removal of one or more toxins from the body. In this way, the composition of the residual fluid remaining in the body, being deficient in said toxin(s), will be different to the composition of fluid within the body before it was contacted by the device. The fluid contacted by the device may be any desirable type of fluid but it is preferred that the fluid is interstitial tissue fluid or at least one component thereof. Said at least one component of the interstitial fluid is preferably selected from the group consisting of water, a uraemic toxin, a metabolic product, a salt and an ion. Alternatively, said at least one component of interstitial fluid may be selected from the group consisting of Retinol Binding Protein, Beta-2-Microglobulin, Parathyroid hormone, Adrenomedullin, Atrial Natriuretic Peptide, Asymmetric dimethylarginine, Indole-3-Acetic Acid, Uric Acid, Homocysteine, Creatine, Creatinine, P-Cresol, Oxalate, Urea and Phosphate.

In a preferred embodiment of the device according to the first, second, third and/or fourth aspects of the present invention there is provided a self-contained cassette, sleeve, bandage or the like of variable size and shape that can be attached to a patient's limb or trunk and be moved daily to different sites. The area of skin accessed for transdermal interstitial fluid removal would be part of a patient's dialysis prescription once the efficiency of the modality is determined. An appropriate rate of fluid removal for a particular patient will depend upon many factors known to the skilled person. For example, a rate of fluid removal of up to around 2000 ml/day may be appropriate, more preferably up to around 1000 ml/day, or up to around 500 ml/day (equivalent to 0.35 ml/minute). An appropriate lower limit for the rate of fluid removal may be around 10 ml/day, more preferably around 50 ml/day and most preferably around 100 ml/day. Periodic replacement of the high capacity absorbent material may be required, e.g. replacement may be required on a weekly, daily or more frequent basis, such as twice, thrice or more frequently each day. In certain preferred embodiments of the present invention the high capacity absorbent material may be reusable following appropriate reconditioning, or may simply be discarded. Every function may be contained within the unit, such that the unit requires no external connections for power or other services, with consequently no constraints on patient mobility or life style. There may be further provided a display on the device to provide an indication of the analytical composition of the extracted fluid (e.g. displaying levels of creatine, lactate, glucose, sodium, potassium, calcium, phosphate and/or other uraemic toxins or metabolites).

The devices according to the first, second, third or fourth aspects of the present invention preferably comprise microneedle arrays of any desirable number of microneedles to suit a particular application. The array may comprise up to around 900 microneedles (optionally in a symmetrical 30×30 arrangement), up to around 625 microneedles (optionally arranged as 25×25), up to around 400 microneedles (optionally in a 20×20 arrangement), up to around 225 microneedles (optionally in a 15×15 arrangement), or up to around 100 microneedles (optionally in a 10×10 arrangement). The needles in the microneedle array can be arranged substantially symmetrically or alternatively non-symmetrically. By way of example, an array consisting of 100 microneedles may incorporate a symmetrical arrangement of 10×10 needles or a non-symmetrical arrangement of 5×20 needles. The spacing between neighbouring needles in the microneedle array may be substantially uniform throughout the array, or it may vary as desired throughout the array. It should be appreciated that a symmetrical array of needles may be arranged such that the spacing between neighbouring needles is uniform throughout the array, or alternatively the spacing may vary. The fact that the needles are arranged symmetrically does not necessitate uniform spacing between needles, even though this might be preferable in certain embodiments. Moreover, the array of microneedles may incorporate a combination of different types of microneedles. Every microneedle within an array provided in a device according to the present invention may have the structure and/or properties of the specified at least one microneedle, or some microneedles may have the specified structure and/or properties while others do not. The array of microneedles may combine microneedles of different heights, inner and/or outer diameters, cross-sectional shapes and spacings between neighbouring microneedles. Each microneedle within an array may have a straight shaft, a regularly tapered shaft, or a combination of a straight section and a tapered section. The or each microneedle may possess a shaft that defines a substantially circular or non-circular cross-section.

In a preferred embodiment of the present invention an electric field may be used to drive the transdermal extraction of interstitial fluid and/or its components in to the high capacity absorbent material. The device according to the first, second, third and/or fourth aspects of the present invention may further comprise positive and negative electrodes connected to a power supply which is operable to provide a reverse iontophoretic gradient between said body fluid and the high capacity absorbent material. Use of an external electric field in this way (often referred to as “reverse iontophoresis”) significantly increases the efficiency of the extraction process. An aspect of the present invention relates to the use of the reverse iontophoresis as an additional selectivity and solute volume modulator for the transdermal filtration modality. Direct current electric field may be supplied by any appropriate source of electrical energy, such as, but not limited to, a battery (e.g. a lithium battery) or a solar powered energy source. By way of example, the electrical energy source can be connected to the transdermal array in the manner shown in FIG. 2, which will be described in greater detail below.

The high capacity absorbent material employed in the devices according to the first, second, third and fourth aspects of the present invention is preferably a hydrogel. The material may comprise polymeric beads and/or a micro-patterned polymeric surface coating. Examples of suitable polymer hydrogels may consist of crosslinked poly ethylene glycol (PEG), acrylate and methacrylate derivatives of polyethylene glycol, polyurethanes having soft segment structures containing polyethylene glycol, poly acrylamide (PAm), polyacrylate salts such as sodium or potassium polyacrylate, polyvinyl pyrrolidone, poly 2-hydroxy ethyl methacrylate (pHEMA), and any derivative or copolymer of said hydrogels. Other hydrogels may be based on crosslinked natural polysaccharides or semi-synthetic cellulose derivatives such as gellan gum, guar gum, xanthan gum, alginate, chitosan, carboxy methyl cellulose, hydroxyl ethyl cellulose, hydroxyl ethyl methyl cellulose, hydroxyl propyl cellulose, hydroxyl propyl methyl cellulose. These materials may be crosslinked alone or in combination with others listed above by reaction with appropriate multifunctional crosslinking agents at elevated temperature. A wide range of crosslinking agents may be used for this purpose including divinyl substituted compounds, such as ethylene dimethacrylate or divinyl benzene, melamine formaldehyde resins, polymers and copolymers of N-methylol acrylamide, and citric acid among the many that will be known to those skilled in the art.

The high capacity absorbent material in the device is preferably substantially dry prior to using the device to remove fluid from said body. This is especially advantageous in embodiments of the present invention where the device is being used in filtration of body fluids, i.e. extraction of relatively large quantities of body fluids. As described previously herein, this process is can be contrasted from purely sampling applications, in which it may be preferred that the absorbent material is wet or partly wet prior to application of the device to the body, to ensure that only very small amounts of fluid are removed, the amount being sufficient for testing, or that substantially no fluid is removed.

It is preferred that the high capacity absorbent material is permeable to molecules having a molecular weight of up to around 50 to 80 kDa, more preferably around 60 to 70 kDa, and most preferably up to approximately the molecular weight of albumin, which is around 67 kDa.

Interstitial fluid is not usually available in quantity for study (as is blood, urine etc.), however, levels of key components in ureamia are known and are shown below in Table I (which refers to uraemic blood). A standard solution of the following components at concentrations found in uraemia includes: retinal binding protein, beta 2 microglobulin, uric acid, creatinine, urea, cations K₊, Na⁺, anions Cl⁻, PO₄⁻.

TABLE 1

| | Molec- | | Normal | Uraemic | Maximum |
|---|---|---|---|---|---|
| Name | ular | | Conc. | Conc. | Conc. |
| (Unit) | Weight | Group | (CN) | (CU) | (CM) |
| Retinol Binding | 21,200 | Protein | <80.00 | 192.00 | 369.20 |
| Protein (mg/L) | | | | | |
| Beta-2- | 11,818 | Protein | <2.00 | 55.00 | 100.00 |
| Microglobulin | | | | | |
| (mg/L) | | | | | |
| Parathyroid | 9,225 | Protein | <0.06 | 1.20 | 2.40 |
| hormone | | | | | |
| (μg/L) | | | | | |
| Adrenomedullin | 5,729 | Protein | 13.20 | 41.80 | 81.20 |
| (ng/L) | | | | | |
| Atrial Natriuretic | 3,080 | Peptide | 28.00 | 202.00 | 436.60 |
| Peptide (ng/L) | | | | | |
| ADMA (mg/L) | 202 | Guanidin | 0.20 | 1.60 | 7.30 |
| Indole-3-Acetic | 175 | Indol | 17.50 | 875.00 | 9076.90 |
| Acid (μg/L) | | | | | |
| Uric Acid (mg/L) | 168 | Purine | <67.20 | 83.40 | 146.70 |
| Homocysteine | 135 | Other | <1.70 | 8.10 | 26.40 |
| (mg/L) | | | | | |
| Creatine (mg/L) | 131 | Guanidin | 9.70 | 134.00 | 235.80 |
| Creatinine (mg/L) | 113 | Guanidin | <12.00 | 136.00 | 240.00 |
| P-Cresol (mg/L) | 108 | Phenol | 0.60 | 20.10 | 40.70 |
| Oxalate (mg/L) | 90 | Other | 0.30 | 4.90 | 7.60 |
| Urea (g/L) | 60 | Other | <0.40 | 2.30 | 4.60 |

Notwithstanding the above, preliminary experiments have been call led out to measure the concentration of the ureamic toxin urea in the plasma, interstitial fluid and induced sweat of a normal subject and a patient with chronic kidney disease (CKD) on peritoneal dialysis. The results are presented below in Table 2.

TABLE 2

| | Control | | | CKD Patient | | |
|---|---|---|---|---|---|---|
| | | In IF via | In induced | | In IF via | In induced |
| Toxin | In Plasma | microdialysis | sweat | In Plasma | microdialysis | sweat |
| Urea(mmol/L) | 7.43 | 4.8 | 6.25 | 16.65 | 22.83 | 22.97 |

The results in Table 2 demonstrate that in the patient suffering from renal failure, the interstitial fluid (IF) collected via conventional microdialysis and the induced sweat contained more urea than the plasma. These results support the view that it is preferred to use the device of the present invention to access the interstitium, rather than the blood compartment, to perform dialysis and related procedures. The results also confirm that sweating can be induced effectively to provide a fluid high in levels of urea. It will be appreciated that combining this knowledge with the ability to produce high capacity absorbent material with selectivity towards urea should enable far greater quantities of urea to be extracted per litre of interstitial fluid and/or sweat using the devices and methods of the present invention than prior art methods and devices.

Chemically reactive functional groups (e.g. amino acids) can also be incorporated in to the polymer which will react with specific target molecules in the interstitial fluid and thereby bind the target molecule to the polymer. Moreover, it is envisaged that biospecific ligands, e.g. selective binding peptides/proteins or enzymes, may be incorporated into the polymer so as to further enhance the selectivity of the high capacity absorbent material.

A further aspect of the present invention relates to a tissue engineered skin covering, which can be implanted at specific sites on a body to express high permeability to water so as to function as a docking site for the attachment of a device according to the first, second, third and/or fourth aspect of the present invention.

In combination with any of the above-defined aspects of the present invention there is provided a vacuum suction device to accelerate the transdermal extraction or removal of water, uraemic toxins, metabolic products, ions and/or salts, which may find particular application in the treatment of fluid overload or uraemia arising from, for example, heart failure and/or renal failure.

Sweat glands are natural excretory organs that mimic the kidneys in the removal of excess salt and water but also in removal of uremic toxins like potassium and urea. The device according to the first, second, third and/or fourth aspects of the present invention is particularly suitable for application to the arms and/or trunk of a patient, which are rich in sweat glands. By incorporating a sweat induction mechanism into the protocol for using the device, it is anticipated that fluid removal rates can be enhanced because sweating mechanism should stimulate excess fluid removal, which can pass through the microneedle bores and be captured by the high capacity absorbent material. Initial sweat induction may also serve to prime the device ready for fluid extraction/filtration. By wetting the bore surfaces of the microneedle and at least partly filling them with fluid (i.e. induced sweat), a continuous column of fluid linking the interstitium and hydrogel can be created, triggering surface tension forces which can initiate fluid removal from the interstitium via the microneedle arrays.

Sweating can be induced thermally or chemically, for example by the administration of pilocarpine that can be delivered transcutaneously to the sweat glands by an iontophoretic current. Initial experiments with pilocarpine at room temperatures have yielded up to 20 microlitres/cm²/hour. This quantity of sweat constitutes an ideal primer for microneedle extraction of interstitial fluid. This output should be improved by incorporating a mechanism to increase the skin temperature to just above body temperature (e.g. 100 F (37.8° C.)) since it is known that over 300 ml of sweat can be obtained from the glands of one arm over a 1 hour period at this temperature, thus underlining the huge potential for temperature induced sweating to aid the removal of quantities of fluid selectively from patients with fluid overload due to various medical conditions, such as kidney failure and cardiac failure.

Methods according to the present invention thus preferably further comprise the step of increasing the rate of fluid flow or loss from the body by stimulating sweat gland secretion of water and ions by the administration of one or more suitable chemical entities (e.g. pilocarpine) and/or the application of an external heat source.

It will be understood that features of the devices according to the first, second, third, and fourth aspects of the present invention may be combined together into a single device, subject to the technical compatibility of the various features. For example, the hollow swellable microneedles of the third aspect may be modified to incorporate the hydrophilic bore lining of the second aspect and/or the microporous bore filling of the first aspect. The hollow hydrophobic microneedles of the second aspect may be modified to incorporate a microporous material within one or more of the bores which already have a hydrophilic lining.

### BRIEF DESCRIPTION OF THE DRAWINGS

Embodiments of the present invention will now be described, by way of example only, with reference to the accompanying drawings in which:

FIG. 1 is a schematic representation of a third embodiment of a device according to the first aspect of the present invention;

FIG. 2 is a schematic representation of a fourth embodiment of a device according to the first aspect of the present invention;

FIG. 3 is a schematic representation of a hollow swellable microneedle for use in a fluid extraction or filtration device according to the third aspect of the present invention;

FIG. 4 is a schematic representation of a solid swellable microneedle not in accordance with the present invention immediately after insertion into a fluid (left) and after having swollen (right) within said fluid;

FIG. 5 is a schematic representation of a fluid extraction or filtration device according to the third aspect of the present invention incorporating a hollow hydrogel microneedle as depicted in FIG. 3;

FIG. 6 is a schematic representation of a fluid extraction or filtration device according to the second aspect of the present invention incorporating a microneedle device having hydrophobic hollow microneedle provided with a hydrophilic coating on the surface of the internal bore of the microneedle;

FIG. 7 is a schematic representation of a fluid extraction or filtration device according to the first aspect of the present invention incorporating a microneedle device having a hollow microneedle filled with a microporous material;

FIG. 8 is a plan view of a schematic illustration of a high capacity absorbent pouch according to an aspect of the present invention; and

FIG. 9 is a cross-sectional view of a schematic illustration of the high capacity absorbent pouch of FIG. 8.

### DETAILED DESCRIPTION OF THE INVENTION

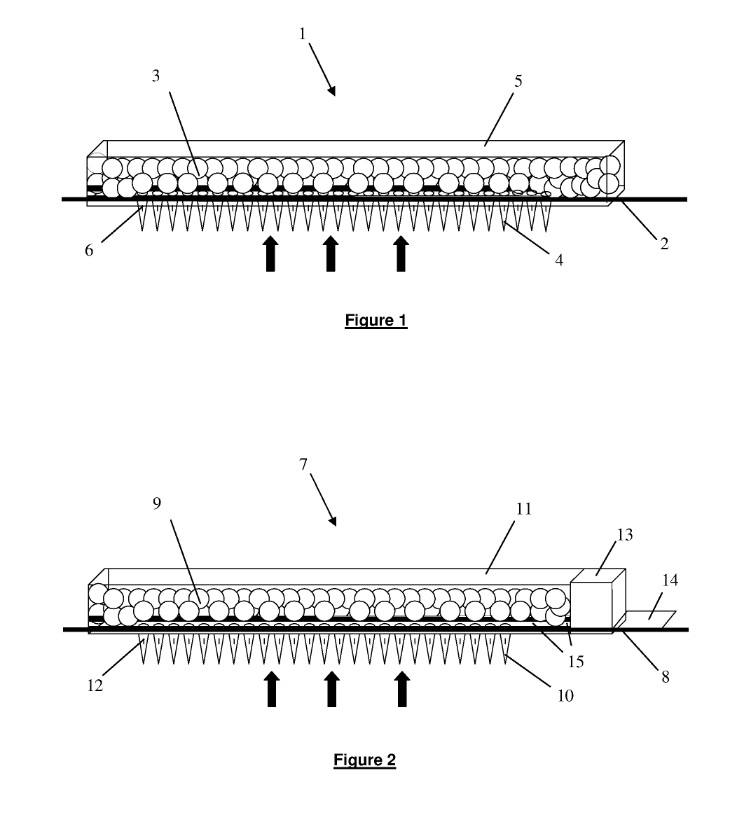

Referring to FIG. 1, there is depicted schematically a device 1, which can be employed in transdermal filtration or purification, according to a preferred embodiment of the present invention. The device 1 is shown positioned on a skin surface 2 of a human body (not shown). The device 1 incorporates a highly absorbent gel matrix 3, which incorporates a loosely cross linked poly sodium acrylate The gel matrix 3 is in fluid communication with a plurality of hollow microneedles 4 formed of a swellable material arranged so as to define a regular array of hollow tapered projections which penetrate the skin's surface 2 as shown in FIG. 1. The gel matrix 3 is retained within a housing 5 which is of sufficient size to accommodate the gel 3 once it has adsorbed up to around 15-25 times its own volume of fluid. Each needle 4 in the array is around 750 μm in height and defines a side pore or inlet aperture 6 of around 50 μm diameter.

When it is desired to use the device 1, a dry gel matrix 3 is placed within the housing 5 and the device 1 then sterilised. The skin surface 2 is wiped with a bacteriocidal solution and the device 1 is then pressed against the skin surface 2 by the application of sufficient pressure to cause the array of microneedles 4 to penetrate the stratum corneum and enter the epidermis.

The swellable microneedles 4 are sufficiently high and the pores 6 appropriately positioned so as to reside within the interstitial tissue fluid bed (not shown). In this way, a fluid conduit is established between the subcutaneous interstitial fluid (not shown) and the gel matrix 3 within the housing 5. By careful selection of the nature of the gel matrix 3 an osmotic and hydrostatic gradient is established between the interstitial fluid and the gel matrix 3 such that water, uraemic toxins, metabolites, ions, salts and the like can be selectively extracted and filtered out of the body in the direction of the arrows in FIG. 1.

Once a cycle has been completed, for example with reference to a predetermined time period or determination of the volume of the gel, the device 1 is then removed from the skin surface 2 and the above described cycle repeated, starting with removal of the swollen gel 3 with a new dry gel 3.

FIG. 2 shows a schematic representation of an alternative embodiment of a device 7 that can also be used for transdermal filtration, purification or dialysis, according to the third aspect of the present invention. The device 7 is very similar to the device 1 shown in FIG. 1 but the device shown in FIG. 2 incorporates an additional component to aid in transdermal movement of target molecules and fluids. The device 7 in FIG. 2 is positioned on a skin surface 8. The device 7 incorporates the same highly absorbent gel matrix 9 as in the device 1 of FIG. 1. The device 7 further incorporates an array of hollow swellable microneedles 10. The gel matrix 9 is retained within a housing 11. Each needle 10 is around 750 μm in height and defines a side pore 12 of around 50 μm diameter.

The new component incorporated in to the device 7 shown in FIG. 2 is a battery 13 connected to positive and negative electrodes 14, 15. Activation of the battery 13 causes a reverse iontophoretic gradient to be established thereby resulting in an electro osmotic flow of fluid from the positive electrode 14 to the negative electrode 15, which enhances the transport of cations and neutral/uncharged species in the direction of the arrows across the skin surface 8 from the interstitial fluid to the gel 9.

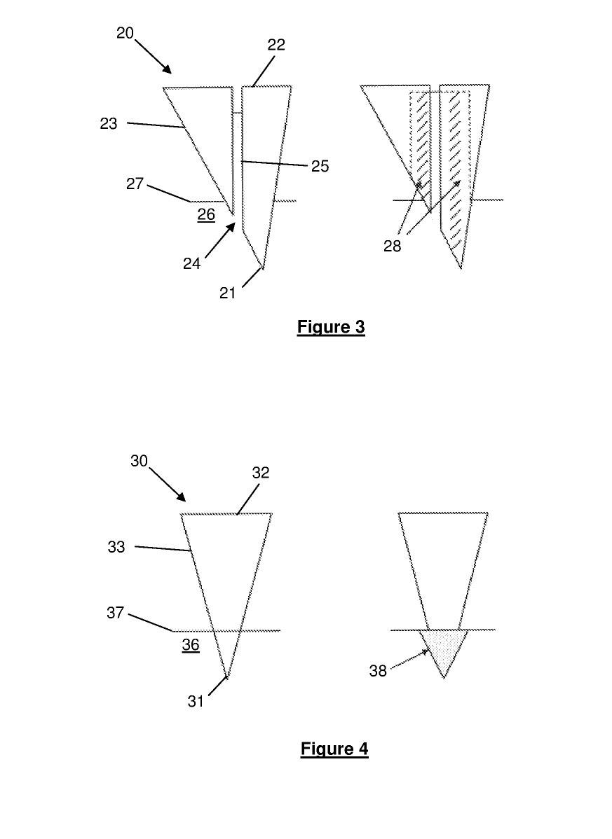

Referring now to FIG. 3 there is shown a hollow swellable microneedle 20 for use in a fluid extraction or filtration device according to the third aspect of the present invention. The swellable microneedle 20 incorporates a tip 21 and a base end 22 connected by a sidewall 23. The sidewall 23 defines an opening 24 to an internal bore 25 which fluidly connects the opening 24 to the base end 22 of the microneedle 20. The lefthand image depicts the microneedle 20 immediately after insertion into a fluid 26 within a body, the surface of which is depicted at 27. The righthand image depicts the structure of the microneedle 20 after areas 28 of the microneedle 20 adjacent to the internal bore 25 have swollen (shown shaded) due to the microneedle 20 being formed from a swellable material.

FIG. 4 schematically illustrates a solid swellable microneedle 30 not in accordance with the present invention with a tip 31, base end 32 and interconnecting sidewall 33. For present purposes the microneedle 30 is referred to as being ‘solid’ by virtue of not defining any internal bores which can cause fluid to flow under capillary action. The microneedle 30 in the lefthand image is shown immediately after insertion into a fluid 36 so that the tip 31 and a small region of the microneedle extending from the tip 31 towards the base end 32 is immersed below the surface 37 of the fluid 36. The righthand image schematically depicts the structure of the microneedle 30 after it has been immersed in the fluid 36 for a sufficient period of time to have absorbed a quantity of fluid. As can be seen, the only section of the solid swellable microneedle 30 that has absorbed fluid is the section 38 that is immersed in the fluid 36, which has become swollen. This swollen section 38 of the microneedle 30 could become plasticised, which could lead to breakage of the microneedle 30 either while implanted or during removal from a body containing fluid.

While solid swellable microneedles of the kind shown in FIG. 4 may be suitable for use in very low volume sampling applications they would not be suitable for use in devices according to aspects of the present invention due to the relatively high level of fluid uptake required to meet a satisfactory level of interstitial fluid toxin removal. The hollow swellable microneedles 20 employed in the device according to the first aspect of the present invention represent a significant improvement to such solid swellable microneedles 30. The presence of one or more bores 25 within each microneedle 20 allows fluid 26 to be removed from a body via the internal bore(s) 25 by capillary action. This capillary action also increases the volume of fluid 26 that can be absorbed into the structure of each microneedle 20 beyond that which can be absorbed by a solid swellable microneedle 30, which is limited to the section 38 of the microneedle 30 that is immersed in the fluid 36 as shown in FIG. 4.

Without wishing to be bound by any particular theory, it is currently believed that the improvement in performance of hollow swellable microneedles 20 as compared to solid swellable microneedles 30 is at least in part due to a reduction in the contact angle of the fluid in which the hollow microneedles 20 are immersed as compared to the solid microneedles 30. The height (h) of fluid rise in a capillary is given by:

h=2γ cos Θ/ρgr a.

where γ is the liquid/air surface tension; Θ is the contact angle; ρ is the liquid density; g is the gravitational force constant; and r is the radius of the capillary.

As the swellable material absorbs fluid the contact angle (Θ) fails to zero giving cos Θ=1, thereby maximising fluid rise in the capillary and significantly increasing fluid absorption by the hollow swellable microneedle 20 as compared to the solid microneedle 30.

Now referring to FIG. 5, there is shown a device 40 according to the third aspect of the present invention which incorporates a hollow swellable microneedle 20 as depicted in FIG. 3 and which takes advantage of the increase in capillary action exhibited by hollow swellable microneedles 20 as compared to solid microneedles 30. In the device 40 shown in FIG. 5 the hollow microneedle 20 has been connected to an intermediate fluid transport layer in the form of wicking 41 which is in fluid communication with a fluid reservoir in the form of a collection pouch 42 constructed of high capacity absorbent material capable of absorbing at least 5 times its own weight in fluid. If the fluid level rise up the internal bore 25 of the hollow swellable microneedle 20 is sufficient the fluid may be brought into contact with the wicking 41 located at the end of the bore 25 defined by the base end 22 of the microneedle 20. The fluid may then pass from the wicking 41 to the collection pouch 42. In the embodiment shown in FIG. 5, transfer of fluid 26 to the collection pouch 42 will continue until the level of fluid in the contact with the bore 25 becomes insufficient to maintain the height of the meniscus in the bore 25 and contact with the collection pouch 42 is broken or the absorptive capacity of the collection pouch 42 is reached. Since a greater proportion of the fluid 26 is passed along the bore 25 by capillary action than which is absorbed by the swellable material of the microneedle 20 the microneedle 20 exhibits a decreased amount of swelling as compared to the solid microneedle 30 the hollow swellable microneedle 20 is less likely than the solid microneedle 30 to become plasticised and break during implantation or removal.

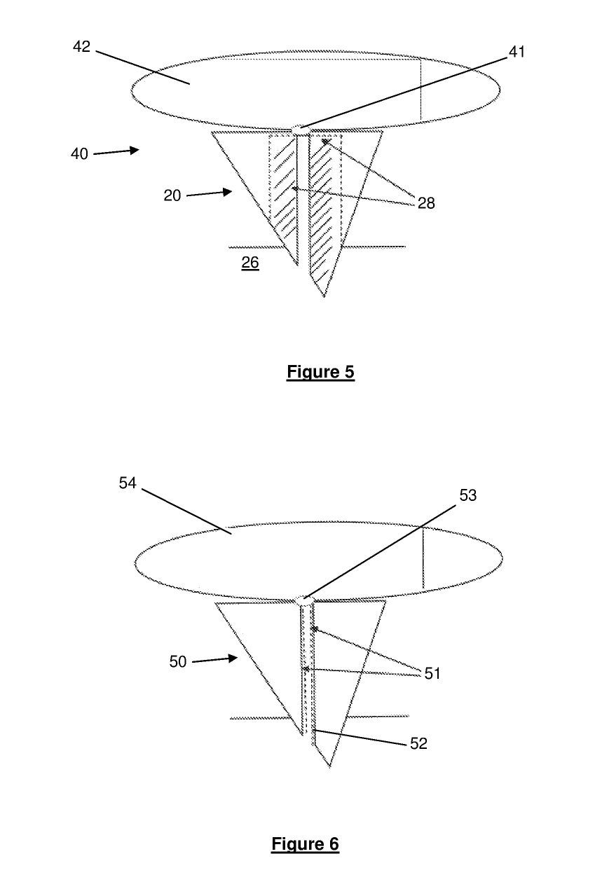

FIG. 6 depicts a hollow microneedle 50 forming part of a microneedle device according to the second aspect of the present invention. The hollow microneedle 50 is manufactured from a strong engineering plastic, polyether-ether-ketone (PEEK), which is sufficiently strong to resist breakage during implantation or removal. Since materials of this kind are hydrophobic and are therefore non-wettable by aqueous media, they exhibit a high contact angle (Θ) with aqueous media, which for the reasons elucidated above, is undesirable. To address this problem the hollow microneedle 50 has been provided with a layer of a hydrophilic material 51 on the internal surface of the microneedle 50 which defines its internal bore 52. As in the device 40 shown in FIG. 4, the end of the bore 52 which opens at the base end of the microneedle 50 is in fluid communication with a layer of wicking which is itself in fluid communication with a collection pouch 54 of a high capacity absorbent material. Providing the layer of hydrophilic material 51 on the wall of the bore 52 in this way causes the contact angle (Θ) of the fluid to tend to zero with the result that cos Θ tends to 1, thereby maximising fluid flow along the bore 52 to the wicking 53 and on to the pouch 54.

The walls of the bore 52 of the microneedle 50 may be rendered hydrophilic using any appropriate technique, such as by treatment with a plasma or by the application of the hydrophilic coating. In the embodiment shown in FIG. 6 a coating of porous silica particles embedded within a hydrophilic polymer binder has been applied to the walls of the bore 52. The particulate nature of the silica confers a very high surface area on the walls of the bore 52 thereby enhancing both the rate of fluid uptake and the height of the fluid meniscus within the bore 52. Any suitable silica particles may be used, but in the present embodiment a Syloid W series silica material (e.g. W300, W500 and W900 from W.R. Grace & Co.) was used. The polymeric binder should be capable of swelling in aqueous media but not dissolve since this would damage the integrity of the hydrophilic layer. In the present embodiment the binder was linear poly(2-hydroxy ethyl methacrylate) (polyHEMA) applied from an alcoholic or aqueous alcoholic mixture at a polymer concentration of 1 to 10% w/w, more preferably 3 to 7% w/w. In an alternative embodiment in which an aqueous application system is desired then chitosan be used in place of polyHEMA. An aqueous solution of the acetate salt of chitosan may be used at a polymer concentration of 0.5 to 3% w/w, more preferably 1 to 2% w/w. As the coating dries the acetate salt of chitosan is converted to the free amine form of the polymer by removal of the acetic acid, thus rendering the coating polymer swellable in aqueous media by insoluble. The ratio of polymer binder to silica particles may be chosen to suit the particular application. A ratio of 2:1 to 1:10 may be used, more preferably a ratio of 1:2 to 1:5 may be used.

To test the performance of a device of the kind shown in FIG. 6 an experiment was performed using an 11×11 array of hollow PEEK microneedles. The array was 1.5 cm×1.5 cm in size. Each microneedle was 700 μm in length (measured from the tip to the base end of the microneedle) and defined a single opening to the internal bore of 90 μm diameter. The walls of the internal bore were coated by applying to the rear of the microneedle array (i.e. adjacent the base end of the microneedles) a dispersion of Syloid W500 particles in a 5% w/w solution of polyHEMA in ethanol. The ratio of Syloid W500:polyHEMA was 2.1. The solution was allowed to permeate through the hollow microneedles. The coating was then dried by the application of a vacuum to the rear of the microneedle array for 1 to 2 minutes.

The coated array of microneedles was inserted through a polyurethane film that was laminated on to a hydrophilic polyurethane foam such that the microneedles were embedded in the foam matrix. The foam containing the embedded microneedle array was then transferred to a Petri dish containing phosphate buffered saline with a dissolved bovine serum albumin content of 25 g/l. The foam was allowed to swell in the albumin-containing solution while a small piece of tissue paper (1 cm×1 cm) was applied to the rear of the microneedle array. After a period of approximately 1 hour the tissue had absorbed a quantity of the albumin-containing solution. A piece of non-woven fabric containing superabsorbent fibres (Oasis 2356 from Technical Absorbents Limited) was applied to the rear of the tissue paper. Fluid rapidly transferred into the fabric, which became saturated in a few minutes. The saturated fabric was removed and replaced with another piece of the same type of fabric, which again absorbed fluid rapidly.

A control test was performed using identical equipment and conditions except that the bores of the PEEK microneedles were not provided with a hydrophilic coating. No fluid transport was observed through the microneedles over a 12 hour period.

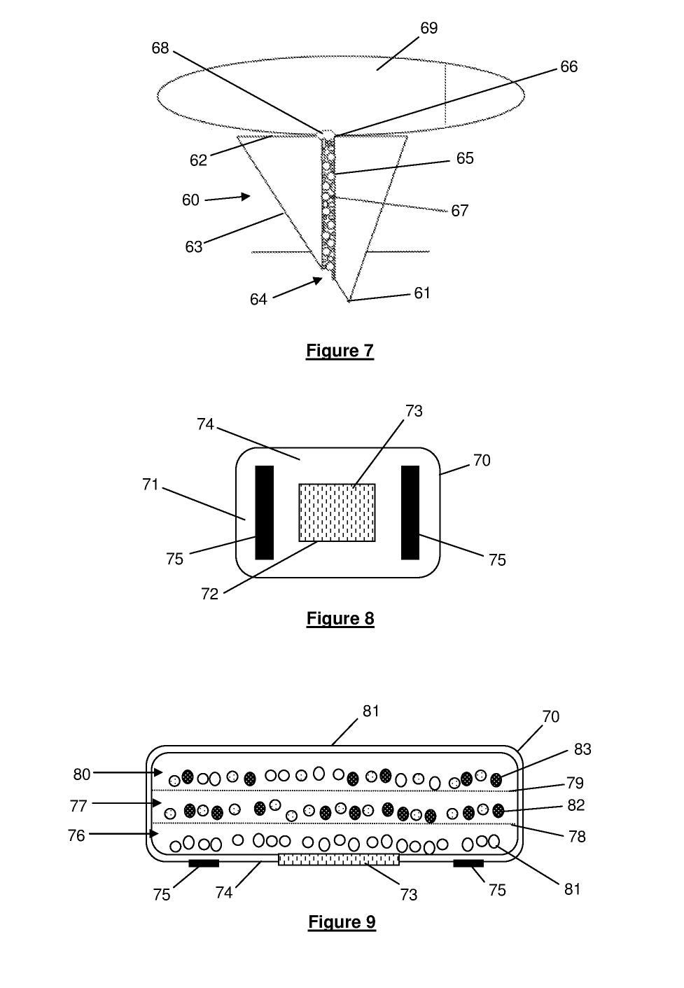

With reference now to FIG. 7, the third aspect of the present invention provides a microneedle device incorporating a hollow microneedle of any desirable material in which the internal bore of the microneedle is provided with a microporous material. A microneedle 60 of this kind is illustrated schematically in FIG. 7. The microneedle 60 defines a tip 61, base end 62 interconnected by a sidewall 63. The sidewall 63 defines an opening 64 to an internal bore 65 which fluidly connects the opening 64 to an orifice 66 defined by the base end 62 of the microneedle 60. In the device shown in FIG. 7 the bore 65 is substantially filled with a microporous material 67. The orifice 66 is closed by a layer of wicking 68 to which is fluidly connected a collecting pouch 69 composed of a high capacity absorbent material.

The microporous material 67 in the bore 65 may be any one or more of a number of different suitable materials. By way of example, an open cell hydrophilic polyurethane foam may be used, or a mixture of two polymers that are incompatible and will phase separate to produce a microporous structure, e.g. a mixture of polyurethane and impact modified polystyrene. Other exemplary materials include polyethylene glycol and polyvinyl alcohol, polyethylene glycol and dextran, or polyethylene glycol and dimethylsiloxane polymers. Another approach is to use a phase inversion process to coagulate a solution to produce a porous matrix. Examples would be the coagulation of a solution of polyurethane in an aprotic solvent with water, or the coagulation of solution of polyether sulfone in dimethyl sulfoxide in an alcoholic solvent. A particularly preferred method is to use a colloidal silica solution that is coagulated via a sol-gel phase inversion process either by changing pH or by the addition of sodium chloride solution, followed by drying to yield a highly porous silica gel network.

To test the performance of a device of the kind shown in FIG. 7 an experiment was performed using an 11×11 array of hollow PEEK microneedles. The array was 1.5 cm×1.5 cm in size. Each microneedle was 700 μm in length (measured from the tip to the base end of the microneedle) and defined a single opening to the internal bore of 90 μm diameter. The internal bore of each microneedle was filled with a microporous material by applying to the rear of the microneedle array a colloidal solution of silica that had been coagulated by mixing 2 parts of the solution with 1 part of 2M sodium chloride solution. The microneedle array was then dried in an oven at 50° for 1 to 2 hours.

The filled microneedles were then tested by inserting them through a polyurethane film that was laminated on to a hydrophilic polyurethane foam such that the microneedles were embedded in the foam matrix. The foam containing the embedded microneedle array was then transferred to a Petri dish containing phosphate buffered saline with a dissolved bovine serum albumin content of 25 g/l. The foam was allowed to swell in the albumin-containing solution while a small piece of tissue paper (1 cm×1 cm) was applied to the rear of the microneedle array. Within a few seconds the tissue had absorbed a quantity of the albumin-containing solution. A piece of non-woven fabric containing superabsorbent fibres (Oasis 2356 from Technical Absorbents Limited) was applied to the rear of the tissue paper. Fluid rapidly transferred into the fabric, which became saturated in a few minutes. The saturated fabric was removed and replaced with another piece of the same type of fabric, which again absorbed fluid rapidly.

A control test was performed using identical equipment and conditions except that the bores of the PEEK microneedles were not filled with a microporous material. No fluid transport was observed through the microneedles over a 12 hour period.

Schematic illustrations of a high capacity absorbent pouch according to an aspect of the present invention are shown in FIGS. 8 and 9. The pouch 70 comprises an outer skin 71 made of a fluid impermeable transparent polymeric material. A section of the polymeric material is produced so as to define an opening 72 over which is placed a layer of wicking material 73. The wicking material 73 is located on a lower side 74 of the pouch which, in use, will face towards the patient undergoing treatment. The lower side 74 is also provided with two strips of pressure sensitive adhesive 75 which, prior to use, are covered by a release paper (not shown) that can be removed shortly before the pouch 70 is to be applied to the patient's body. As can be seen in FIG. 9, the internal construction of the pouch 70 incorporates three compartments located one on top of the other. The lowermost compartment 76 is located nearest to the lower side 74 of the pouch 70. The interface between the lowermost compartment 76 and a middle compartment 77 is defined by a further layer of wicking material 78. Another layer of wicking material 79 defines the interface between the middle compartment 77 and the uppermost compartment 80 which is nearest an upper side 81 of the pouch 70 that will face away from the patient when the pouch 70 is applied to the patient. While the specific embodiment shown in FIGS. 8 and 9 incorporates three compartments 76, 77, 80 separated by two internal layers of wicking material 78, 79 it will be appreciated that the pouch may define any suitable number of compartments provided in any desirable arrangement. For example, the pouch may incorporate just a single compartment in which case no internal wicking layers would be needed. Alternatively, the pouch may incorporate two, three, four or more compartments which may be arranged one on top of the other in a similar manner to the arrangement shown in FIG. 9, or which may be arranged side-by-side, diagonally disposed with respect to one another, or any other desirable arrangement. Moreover, where two or more compartments are provided, the compartments may all be of the same size and shape, or they may differ in size and/or shape.

In FIG. 9, the lowermost compartment 76 of the pouch 70 is provided with a first layer of absorbent material 81 made up of a superabsorbent material and porous silica. A second layer of absorbent material 82 is then provided in the middle compartment 77 made up of particles of clay and activated carbon. The uppermost compartment 80 is provided with a third layer of absorbent material 83 made up of a mixture of the absorbents provided in the first and second layers of absorbent material.

As mentioned above, the interfaces between the three compartments 76, 77, 80 are defined by layers of wicking material 78, 79. As a result, in use, fluid captured by the first layer of absorbent material 81 is drawn out of the first layer of absorbent material Si by wicking material 78 from which it can then be absorbed by the second layer of absorbent material 82. Subsequently, as the volume of fluid absorbed by the second layer of absorbent material 82 increases the wicking material 79 in between the middle and uppermost compartments 77, 80 will start to draw fluid out of the second layer of absorbent material 82 and pass it to the third layer of absorbent material 83. In this way, all three compartments 76, 77, 80 are in fluid communication with the source of the fluid enabling very large volumes of fluid to be safely removed from a patient in need of such treatment quickly enough to reduce or effectively eliminate the chance of maceration of the skin occurring.

One aspect of the present invention relates to the direct application of a high capacity absorbent material to an array of holes punctured into the skin of a patient requiring treatment using a suitably configured array of microneedles. It will be appreciated that the pouch described above in relation to FIGS. 8 and 9 is eminently suitable for such use simply by ensuring that the external layer of wicking material 73 in the outer skin 71 of the pouch 70 is located on the area of the patient's skin that has been punctured.

Various other aspects of the present invention employ different designs of microneedle devices to establish fluid flow paths through a patient's skin along which interstitial fluid can flow from areas of tissue retaining such fluid. The pouch 70 described above with reference to FIGS. 8 and 9 can be used with each of the different designs of microneedle simply by securing an array of the microneedles to the external layer of wicking material 73 in the outer skin 71 of the pouch 70. This then defines a path for fluid to flow out of the patient's tissue along the microneedles and into the layers of absorbent material 81, 82, 83 within the pouch 70.

It will be appreciated that the novel features of the different embodiments of the devices described above with reference to FIGS. 1 to 3 and 5 to 7 may be employed individually as described above or any two or more novel features may be employed together in the same device. By way of example, the walls of the internal bore defined by the hydrogel microneedle depicted in FIGS. 3 and 5 may be treated to increase their hydrophilicity akin to the microneedles of the device depicted in FIG. 6. Moreover, the same hydrogel microneedles may have a microporous material provided in the internal bore of each microneedle either in combination with the hydrophilic wall treatment or not. As a further example, the hydrophobic PEEK microneedles described above with reference to FIG. 6 which were provided with a hydrophilic lining to their internal bores may also be modified to incorporate a microporous material within those bores. Alternatively, in embodiments of microneedles which define multiple internal bores, any one or more of the bores may be modified to incorporate hydrophilic internal walls, while any one or more of the other bores may be provided with a microporous material.

1.-66. (canceled)

67. A method for removing fluid from a body, the method comprising:
contacting fluid from the body with an array of microneedles to establish a fluid path for fluid to flow out of said body; and
applying vacuum suction to the fluid flow path such that fluid flows out of the body under the influence of said vacuum suction.

68. The method according to claim 67, wherein the vacuum provides a suction pressure of around 75-250 mmHg.

69. The method according to claim 67, wherein the method further comprises providing a high capacity absorbent material to collect fluid flowing out of the body.

70. The method according to claim 69, wherein the high capacity absorbent material is capable of imbibing at least 5 times the weight of fluid per unit weight of absorbent.

71. The method according to claim 69, wherein the fluid flows from the swollen tissue to the high capacity absorbent material via said fluid flow path at a flow rate of at least around 0.007 ml/min.

72. The method according to claim 69, wherein the fluid flows from the swollen tissue to the high capacity absorbent material via said fluid flow path at a flow rate of around 0.035 to around 0.7 ml/min.

73. The method according to claim 69, wherein the high capacity absorbent material comprises a polymer.

74. The method according to claim 69, wherein the high capacity absorbent material comprises a hydrogel.

75. The method according to claim 67, wherein the microneedles are removed from the body prior to application of the vacuum.

76. The method according to claim 67, wherein the microneedles are solid.

77. A method of removing fluid from the body of a patient suffering from a condition associated with excess fluid, wherein the method comprises:
contacting fluid from the body with an array of microneedles to establish a fluid path for fluid to flow out of said body; and
applying vacuum suction to the fluid flow path such that fluid flows out of the body under the influence of said vacuum suction;
wherein the condition is selected from the group consisting of: uraemia, salt and water overload, oedema and renal failure.

78. The method according to claim 77, wherein the fluid contains a target species.

79. The method according to claim 78, wherein the target species is selected from the group consisting of water, a uraemic toxin, a metabolic product, a salt and an ion.

80. A method for treating oedema by the removal of interstitial fluid from an area of oedema in a body, the method comprising:
inserting one or more arrays of microneedles into tissue swollen as a result of oedema;
removing the microneedles to establish a fluid path for fluid within the oedema to flow from the swollen tissue; and
applying vacuum suction to the fluid flow path such that fluid flows out of the swollen tissue under the influence of said vacuum suction.

81. The method according to claim 80, wherein the vacuum provides a suction pressure of around 75-250 mmHg.

82. The method according to claim 80, wherein the method further comprises providing a high capacity absorbent material to collect fluid flowing out of the swollen tissue.

83. The method according to claim 82, wherein the high capacity absorbent material is capable of imbibing at least 5 times the weight of fluid per unit weight of absorbent.

84. The method according to claim 82, wherein the fluid flows from the tissue to the high capacity absorbent material via said fluid flow path at a flow rate of at least around 0.007 ml/mm.

85. The method according to claim 82, wherein the fluid flows from the tissue to the high capacity absorbent material via said fluid flow path at a flow rate of around 0.035 to around 0.7 ml/min.

86. The method according to claim 82, wherein the high capacity absorbent material comprises a polymer or hydrogel.
